(19) United States
(12) Patent Application Publication
(10) Pub. No.: US 2018/0310818 A1
(43) Pub. Date: Nov. 1, 2018

(54) IMAGE PROCESSING APPARATUS, IMAGE PROCESSING METHOD, AND OPTICAL INTERFERENCE TOMOGRAPHIC APPARATUS

(71) Applicant: CANON KABUSHIKI KAISHA, Tokyo (JP)

(72) Inventors: Makoto Fukuhara, Yokohama-shi (JP); Toshiharu Sumiya, Hiratsuka-shi (JP)

(21) Appl. No.: 15/772,027

(22) PCT Filed: Oct. 12, 2016
(86) PCT No.: PCT/JP2016/004555

§ 371 Date: Apr. 27, 2018

(30) Foreign Application Priority Data
Oct. 30, 2015 (JP) 2015-215220

(51) Int. Cl. A61B 3/00; A61B 3/10; G06T 7/11

(52) U.S. Cl. CPC A61B 3/0025; A61B 3/0058; A61B 3/102; G06T 2207/30041; G06T 2207/10101; G06T 2207/20021; G06T 7/11

(57) ABSTRACT

An image processing apparatus that processes a plurality of tomographic signals corresponding to light of mutually different polarization, acquired from an object using light interference, extracts candidate regions for depolarization regions of the object in a retardation image of the object, based on retardation values of the object obtained using the plurality of tomographic signals, and extracts depolarization regions of the object in an image indicating uniformity of polarized light in candidate regions, based on a value indicating uniformity of polarized light of the candidate regions, obtained using the plurality of tomographic signals of the candidate regions.

### TECHNICAL FIELD

The present invention relates to an image processing apparatus and image processing method for processing polarization sensitive tomographic images of an object, and an optical interference tomographic apparatus for shooting tomographic images of the object using interference light.

### BACKGROUND ART

In recent years, there have been attempts in the field of ophthalmic equipment at developing optical interference tomographic apparatuses that use optical coherence tomography (hereinafter “OCT”), capable of imaging optical characteristics, movement, and so forth, of fundus tissue. One type of such an OCT apparatus is a polarization-sensitive OCT apparatus, where imaging is performed using polarization characteristics (retardation and orientation, and depolarization), which are optical characteristics of the fundus tissue. Retardation and orientation are indices representing the polarization anisotropy (birefringence) of the object. The degree of anisotropy can be visualized by retardation, and the direction of the optical axis can be visualized by orientation. Polarization anisotropy occurs because of anisotropy in the refractive index of fibrous matter making up the tissue, for example. Depolarization is an index representing the degree of depolarization by the object. It is thought that depolarization is due to the direction and phase of polarized light randomly changing at the time of measurement light reflecting off tissue having ultrastructures such as melanin, for example (see NPL 1).

Polarization-sensitive OCT can form polarization sensitive tomographic images using polarization characteristics, to distinguish and segment fundus tissue. A polarization-sensitive OCT apparatus uses light that has been modulated to circularly-polarized light as measurement light for observing a specimen, performs detection by dividing interference light into two mutually-orthogonal polarized light components, and generates a polarization sensitive tomographic image. Retardation (degree of birefringence) and orientation (direction of optical axis) can be calculated as a polarization sensitive tomographic image, indicating the phase difference between the two orthogonal polarized light components. A Stokes vector is obtained from the intensity and phase difference of the polarized light components. It is known that polarized light is depolarized at particular tissue in the fundus, so retardation and Stokes vectors become uneven. The degree of depolarization can be obtained by calculating a degree of polarization uniformity (DOPU) that indicates the uniformity of polarized light, from the Stokes vectors (see NPL 2). At this time, windows are optionally set in the obtained tomographic image, and the DOPU is calculated for each window. The DOPU is a numeric value representing the uniformity of polarized light that is near 1 where polarization is maintained, but is smaller than 1 where depolarized. Calculating uniformity within the window by DOPU enables stable evaluation of depolarization.

In the structure within the retina, the optic nerve fiber layer (NFL) has polarization anisotropy. There is expectation that observing the NFL may assist in diagnosis of disorders relating to the optic nerve fiber layer (e.g., glaucoma). Also, in the structure within the retina, the retinal pigment epithelium (RPE) layer has a depolarizing nature. The RPE layer can be visualized by obtaining regions that depolarize (depolarization regions), and there is expectation that this may assist in diagnosis of disorders relating to abnormalities of the RPE layer (e.g., age-related macular degeneration).

### CITATION LIST

### Non Patent Literature

NPL 1: B. Baumann, et al, “Polarization sensitive optical coherence tomography of melanin provides intrinsic contrast based on depolarization”, Biomedical OPTICS EXPRESS, Vol. 3, No. 7, P 1670-1683 (2012)
NPL 2: E. Gotzinger, et al, “Retinal pigment epithelium segmentation by polarization sensitive optical coherence tomography”. OPTICS EXPRESS, Vol. 16, No. 21, P 16410-16422 (2008)

### SUMMARY OF INVENTION

### Solution to Problem

According to an aspect of the present invention, an image processing apparatus that processes a plurality of tomographic signals corresponding to light of mutually different polarization, acquired from an object using light interference, includes: a first computing unit configured to compute distribution of retardation values of the object, based on the plurality of tomographic signals; a first extracting unit configured to extract candidate regions for depolarization regions of the object in a retardation image of the object, based on the calculated distribution of retardation values; a second computing unit configured to compute distribution of a value indicating uniformity of polarized light in the extracted candidate regions, based on the plurality of tomographic signals in the extracted candidate regions; and a second extracting unit configured to extract depolarization regions of the object in an image indicating uniformity of polarized light in the extracted candidate regions, based on distribution of the calculated value indicating uniformity of polarized light.

According to an aspect of the present invention, an image processing apparatus that processes a plurality of tomographic signals corresponding to light of mutually different polarization, acquired from an object using light interference, includes: a first extracting unit configured to extract candidate regions for depolarization regions of the object in an image indicating phase difference of polarized light of the object, based on a value indicating phase difference of polarized light of the object obtained using the plurality of tomographic signals; and a second extracting unit configured to extract depolarization regions of the object in an image indicating uniformity of polarized light in the extracted candidate region, based on a value indicating uniformity of polarized light in the extracted candidate region, obtained using the plurality of tomographic signals of the extracted candidate regions.

Further features of the present invention will become apparent from the following description of exemplary embodiments with reference to the attached drawings.

### BRIEF DESCRIPTION OF DRAWINGS

FIG. 1 is a diagram illustrating a shooting flow of an image processing apparatus according to an embodiment.

FIG. 2 is a diagram illustrating the image processing apparatus according to the embodiment.

FIG. 3A is a diagram illustrating a tomographic image shot by the image processing apparatus according to the embodiment.

FIG. 3B is a diagram illustrating a tomographic image shot by the image processing apparatus according to the embodiment.

FIG. 3C is a diagram illustrating a tomographic image shot by the image processing apparatus according to the embodiment.

FIG. 3D is a diagram illustrating a tomographic image shot by the image processing apparatus according to the embodiment.

FIG. 3E is a diagram illustrating a tomographic image shot by the image processing apparatus according to the embodiment.

FIG. 4 is a diagram illustrating retardation distribution in a depolarization layer, acquired by the image processing apparatus according to the embodiment.

FIG. 5A is a diagram illustrating retardation distribution in a non-depolarization layer, acquired by the image processing apparatus according to the embodiment.

FIG. 5B is a diagram illustrating retardation distribution in a depolarization layer, acquired by the image processing apparatus according to the embodiment.

FIG. 6A is a diagram illustrating a map according to the embodiment.

FIG. 6B is a diagram illustrating a map according to the embodiment.

FIG. 6C is a diagram illustrating a map according to the embodiment.

FIG. 6D is a diagram illustrating a map according to the embodiment.

### DESCRIPTION OF EMBODIMENT

In polarization-sensitive OCT, there is a great amount of analysis data to obtain retardation, orientation, DOPU, and so forth from polarization information, and analysis processing takes time. It has thus been an issue for polarization-sensitive OCT to reduce the amount of time from shooting to displaying analysis results. DOPU calculation has required a great amount of time in particular. The reason is that there is the need to set windows for all regions of the tomographic image, and also there is a need to calculate a Stokes vector for each pixel using the intensity ratio and phase difference of two orthogonal polarized light components. Another reason is that DOPU calculation is performed based on values obtained by averaging each factor (Stokes parameter) of Stokes vectors calculated for each pixel in each window.

It has been found desirable to reduce the amount of time needed to extract depolarization regions.

An image processing apparatus according to an aspect of the present invention includes a first extracting unit configured to extract candidate regions for depolarization regions of an object in an image (retardation image) indicating phase difference of polarized light of the object, based on a value (retardation value) indicating phase difference of polarized light of the object, obtained using a plurality of tomographic signals corresponding to light of mutually different polarization, acquired from an object using light interference, and a second extracting unit configured to extract depolarization regions of the object in an image indicating uniformity of polarized light in the extracted candidate regions, based on a value indicating uniformity of polarized light of the candidate regions obtained using a plurality of tomographic signals of the candidate region.

The first extracting unit preferably extracts a region, where the value indicating the phase difference of polarized light that has been computed in the image indicating the phase difference of polarized light is 35° or greater, for example, as the candidate region. The image processing apparatus according to the present embodiment preferably includes a first computing unit configured to compute distribution of a retardation value of the object, based on the plurality of tomographic signals. The image processing apparatus according to the present embodiment also preferably includes a second computing unit configured to compute distribution of a value indicating uniformity of polarized light in the extracted candidate regions, based on the plurality of tomographic signals in the extracted candidate regions.

Accordingly, the amount of time needed to extract depolarization regions can be reduced.

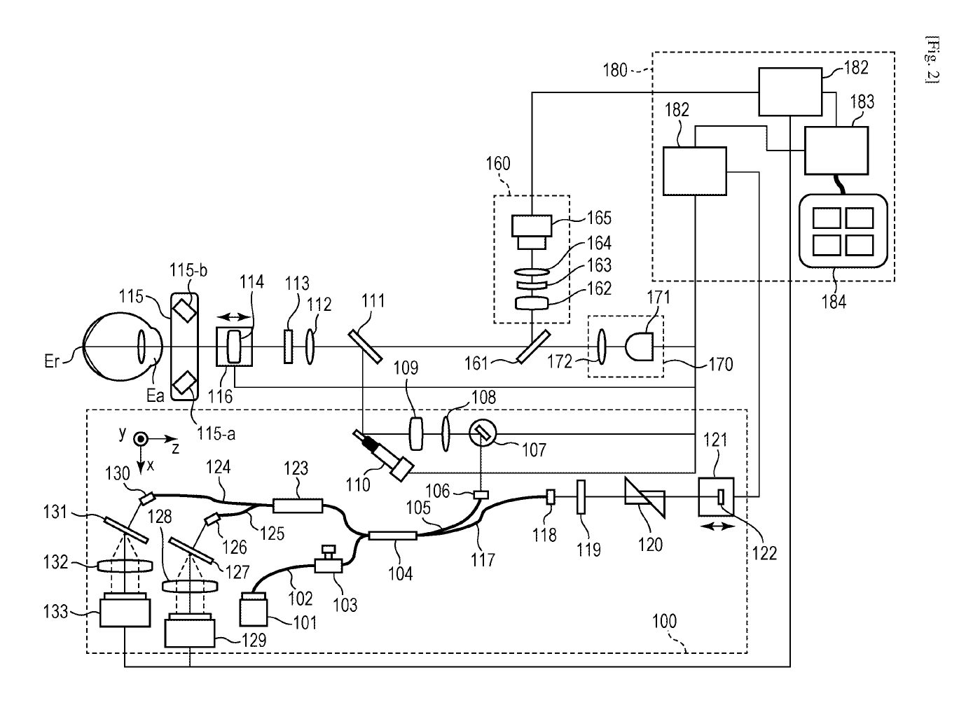

An embodiment of the present invention will be described by way of the drawings. FIG. 2 is a diagram illustrating an optical interference tomographic apparatus internally including the image processing apparatus according to the present embodiment, or communicably connected to the image processing apparatus according to the present embodiment. In the present embodiment, an eye to be examined is the object, and description will be made regarding an optical interference tomographic apparatus (ophthalmic equipment) that obtains images of the object. The optical interference tomographic apparatus is a spectral-domain polarization-sensitive OCT (hereinafter “SDPS-OCT”), as illustrated in FIG. 2. The optical interference tomographic apparatus includes an interference optical meter 100, an anterior ocular segment imaging unit 160, an interior fixation lamp 170, and a control device 180. Alignment of the apparatus is performed using an anterior ocular segment image of the object as observed by the anterior ocular segment imaging unit 160. After the alignment has been completed, the interior fixation lamp 170 is turned on, and in a state with the eye to be examined gazing at the interior fixation lamp 170, fundus photography is performed by the interference optical meter 100.

### Interference Optical Meter 100

The configuration of the interference optical meter 100 will be described. A light source 101 is a super luminescent diode (SLD) light source which is a low-coherence light source. The light source 101 emits light having a center wavelength of 850 nm and a bandwidth of 50 nm. Although an SLD is described as being used for the light source 101, any light source capable of emitting low-coherence light may be used, such as an amplified spontaneous emission (ASE) light source or the like. The light emitted from the light source 101 is guided to a polarization-maintaining fiber coupler 104 via a polarization-maintaining fiber 102 and polarization controller 103, and branches into measurement light and reference light.

The polarization controller 103 is for adjusting the state of polarization of the light emitted from the light source 101 so as to be adjusted to linearly-polarized light. In the case of the present embodiment, polarization is adjusted in a direction perpendicular to a reference polarization direction of branching in a polarization beam splitter in a later-described fiber coupler 123. Although the polarization controller 103 is described as an inline polarization controller in the present embodiment, this is not restrictive. The polarization controller 103 may be a paddle polarization controller having multiple paddles, for example. Alternatively, the polarization controller 103 may be a polarization controller where a quarter-wave plate and half-wave plate have been combined.

The branching ratio at the polarization-maintaining fiber coupler 104 is 90 (reference light) to 10 (measurement light). The branched measurement light is emitted as parallel light from a collimator 106 via a polarization-maintaining fiber 105. The emitted measurement light passes through an X-scanner 107, lenses 108 and 109, and a Y-scanner 110, and reaches a dichroic mirror 111. The X-scanner 107 and Y-scanner 110 are made up of galvano mirrors that scan the measurement light in the horizontal direction and vertical direction at a fundus Er. The X-scanner 107 and Y-scanner 110 are controlled by a driving control unit 181, and can scan a region of the fundus Er by measurement light.

The dichroic mirror 111 has properties where light of 800 nm to 900 nm is reflected, and other light is transmitted. Measurement light reflected at the dichroic mirror 111 passes through a lens 112. The phase thereof is shifted 90° by passing through a quarter wave plate 113 inclined at a 45° angle, and the polarization is controlled to be circularly-polarized light. Note that the light entering the eye to be examined is light of which polarization has been controlled to be circularly-polarized light, by the quarter wave plate 113 being inclined at a 45° angle, but may not be circularly-polarized light at the fundus Er, depending on the properties of the eye to be examined. Accordingly, a configuration has been made where the inclination of the quarter wave plate 113 can be fine-tuned, by control of the driving control unit 181.

The measurement light of which the polarization has been controlled to be circularly-polarized light is focused on a retina layer of the fundus Er by a focus lens 114 on a stage 116, via an anterior ocular segment Ea which is the object. The measurement light cast upon the fundus Er is reflected/scattered at each retina layer, and returns on the optical path to the polarization-maintaining fiber coupler 104.

The reference light which has branched at the polarization-maintaining fiber coupler 104 passes through a polarization-maintaining fiber 117 and is emitted from a collimator 118 as parallel light. The emitted reference light is subjected to polarization control by a quarter wave plate 119 inclined at a 22.5° angle. The reference light passes through a dispersion compensation glass 120, is reflected at a mirror 122 on a coherence gate stage 121, and returns to the polarization-maintaining fiber coupler 104. The reference light passes through the quarter plate 119 twice, whereby linearly-polarized light returns to the polarization-maintaining fiber coupler 104. In the case of the present embodiment, the polarization of the light is adjusted to be linearly polarized light with a 45° inclination as to a reference polarization direction of branching at the later-described fiber coupler 123. The coherence gate stage 121 is controlled by the driving control unit 181 to deal with difference in the axial length of the eye of the object, and so forth.

The reflected light of the measurement light which has returned to the polarization-maintaining fiber coupler 104 and the reference light are multiplexed to form interference light, which is input to the fiber coupler 123 in which a polarization beam splitter is built in, and split into p-polarized light and s-polarized light which have different polarization directions, at a branching ratio of 50 to 50. The p-polarized light passes through a polarization-maintaining fiber 124 and a collimator 130, is dispersed at grating 131, and received at a lens 132 and line camera 133. In the same way, the s-polarized light passes through a polarization-maintaining fiber 125 and a collimator 126, is dispersed at grating 127, and received at a lens 128 and line camera 129. Note that the grating 127 and 131, and line cameras 129 and 133 are positioned in accordance to each polarization direction. The light received at each of the line cameras 129 and 133 is output as electric signals in accordance to the intensity of light, and received at a signal processing unit 182.

### Anterior Ocular Segment Imaging Unit 160

The anterior ocular segment imaging unit 160 will be described. The anterior ocular segment imaging unit 160 illuminates the anterior ocular segment Ea using an illumination light source 115 including LEDs 115a and 115b which emit illumination light having a wavelength of 1000 nm. The light reflected at the anterior ocular segment Ea passes through the focus lens 114, quarter wave plate 113, lens 112, and dichroic mirror 111, and reaches a dichroic mirror 161. The dichroic mirror 161 has properties where light of 980 nm to 1100 nm is reflected, and other light is transmitted. The light reflected at the dichroic mirror 161 passes through lenses 162, 163, and 164, and is received at an anterior ocular segment camera 165. The light received at the anterior ocular segment camera 165 is converted into electric signals, and received at the signal processing unit 182.

### Interior Fixation Lamp 170

The interior fixation lamp 170 will be described. The interior fixation lamp 170 has a display unit 171 and a lens 172. The display unit 171 includes multiple light-emitting diodes (LEDs) arrayed in a matrix. The lighting position of the LEDs is changed in accordance with the region to be shot, under control of the driving control unit 181. Light from the display unit 171 is guided to the eye via the lens 172. The light emitted from the display unit 171 has a wavelength of 520 nm, and a desired pattern is displayed by the driving control unit 181.

### Control Device 180

The control device 180 will be described. The control device 180 includes the driving control unit 181, the signal processing unit 182, a control unit 183, and a display unit 184. The driving control unit 181 controls each part as described above. The signal processing unit 182 generates images based on signals output from each of the line cameras 129 and 133, and anterior ocular segment camera 165. The signal processing unit 182 also analyzes generated images, and generates visualization information of the analysis results. Details of generating images and so forth will be described later. The control unit 183 controls the overall optical interference tomographic apparatus, and also displays images and the like generated at the signal processing unit 182 on a display screen of the display unit 184. The display unit 184 displays various types of information under control of the control unit 183, for example. The display unit 184 here is a liquid crystal display or the like. The image data generated at the signal processing unit 182 may be transmitted to the display control unit 183 by cable, or wirelessly. In this case, the display control unit 183 can be deemed to be an image processing apparatus. The control unit 180 is made up of a central processing unit (CPU), read-only memory (ROM), random access memory (RAM), and the like. Later-described functions and processing of the control unit 180 are realized by the CPU reading out and executing programs stored in the ROM or the like.

### Image Processing Method

Generating and analyzing images by the signal processing unit 182 will be described next.

### Generating Tomographic Signals

The signal processing unit 182 performs reconstruction processing commonly used in SD-PS-OCT on interference signals input from the line cameras 129 and 133, thereby generating tomographic signals. First, the signal processing unit 182 removes fixed pattern noise from the interference signals. Removal of the fixed pattern noise is performed by extracting the fixed pattern noise by averaging multiple A-scan signals that have been detected and subtracting the fixed pattern noise from the input interference signals. Next, the signal processing unit 182 converts the interference signals from wavelength to wavenumber, and performs Fourier transform, thereby generating tomographic signals. Performing the above processing on the interference signals of two polarization components generates two tomographic signals A_{H} and A_{V}, and phases Φ_{H} and Φ_{V}, based on the polarization components.

### Generating Luminance Image

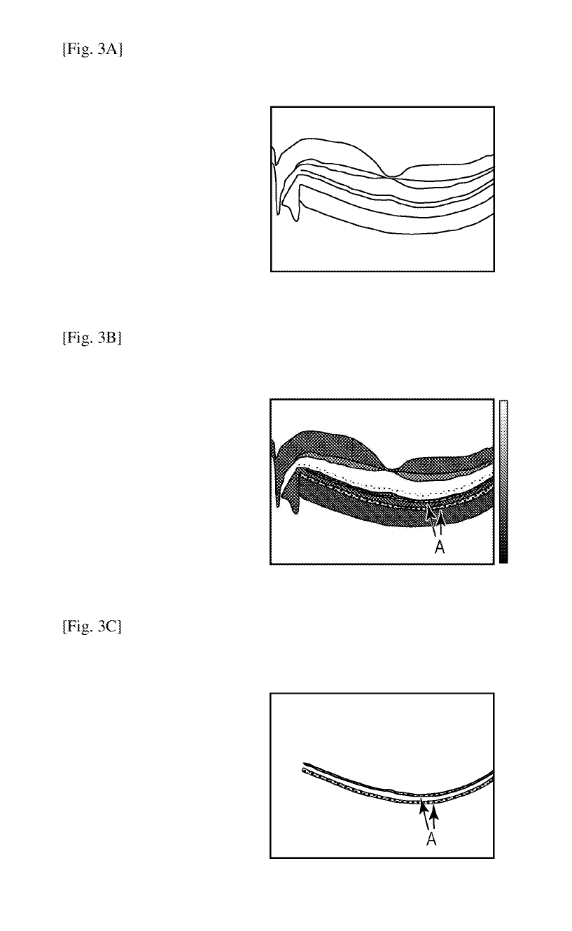

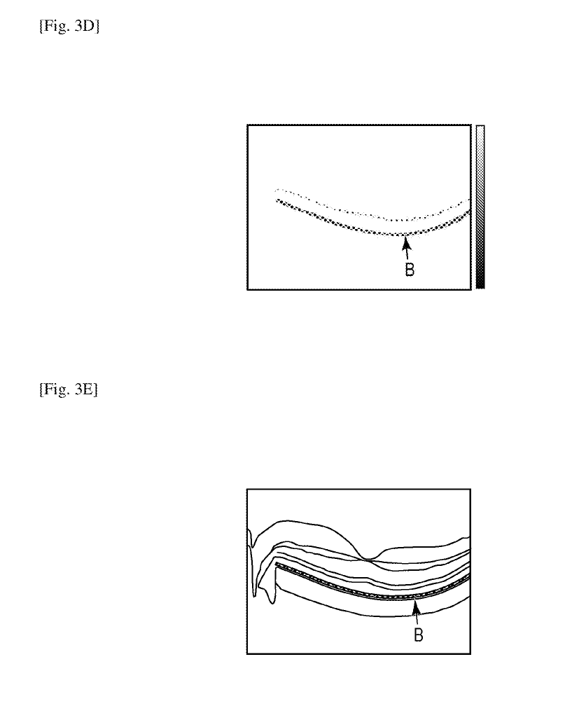

The signal processing unit 182 generates tomographic luminance images from the two tomography signals described above. The signal processing unit 182 arranges the tomographic signals synchronously with driving of the X-scanner 107 and Y-scanner 110, thereby generating two tomographic images based on each polarization component (also referred to as a tomographic image corresponding to first polarized light and a tomographic image corresponding to second polarized light). The tomographic luminance images are basically the same as tomographic images in conventional OCT. A pixel value r thereof is calculated from tomography signals A_{H} and A_{V} obtained from the line cameras 129 and 133, by Expression (1). FIG. 3A illustrates an example of a luminance image of a macular area.

[Math.1]

r=\sqrt{A_{H}²+A_{V}²} Expression (1)

### Generating Retardation Image

Next, generating of a retardation image, which is an example of an image indicating phase difference of polarized light, will be described. The signal processing unit 182, which is an example of a first computing unit, generates retardation images from tomographic signals of mutually orthogonal polarization components. A value δ of each pixel of the retardation image is a value where the phase difference between the vertical polarization component and horizontal polarization component has been made into a numerical value, at the position of each pixel making up the tomographic image. The value δ is calculated from the amplitude of the tomography signals A_{H} and A_{V} by Expression (2).

δ=arctan(A_{V}/A_{H}) Expression (2)

FIG. 3B illustrates an example of a retardation image of the macular area generated in this way (also referred to as a tomographic image indicating phase difference of polarized light), and can be obtained by performing calculation according to Expression (2) on each B-scan image. FIG. 3B shows portions where phase difference occurs in the tomographic image, where dark portions in gradient indicate a small value for the phase difference, and light portions in gradient indicate a great value for the phase difference. The gradation bar at the right side in FIG. 3B represents values of 0 through 900 for retardation. Generating a retardation image enables layers with birefringence to be comprehended. In the structure within the retina, the NFL exhibits a unique birefringence.

Retardation in a case where depolarization of interference light has occurred will be described. It is thought that depolarization is due to reflection at ultrastructures in the tissue (melanin, for example). In a depolarizing region, polarization changes at the time of measurement light reflecting at the boundary face of the ultrastructures. The way in which the polarized light changes differs depending on the reflection surface, so the reflected light has different polarized light non-uniformly (randomly) mixed. This means that the amplitude of the polarization components in the reflected light are non-uniformly (randomly) mixed. The way in which depolarization is exhibited changes depending on the relationship between the magnitude of ultrastructures reflecting the measurement light, and the resolution of the shooting apparatus.

In a case where the resolution of the shooting apparatus is low in comparison with the reflection at the ultrastructures, the non-uniformly polarized light is observed in an averaged manner. There is no bias in the observed polarized light components, so the intensity of the mutually orthogonal polarization components branched at the polarization beam splitter is equal (A_{V}=A_{H}). Accordingly, the retardation calculated by Expression (2) is a constant value such as shown in Expression (3).

δ=arctan(A_{V}/A_{H})=tan−1(1)=45° Expression (3)

Retardation cannot be defined in a depolarization region, meaning that inaccurate values are being calculated.

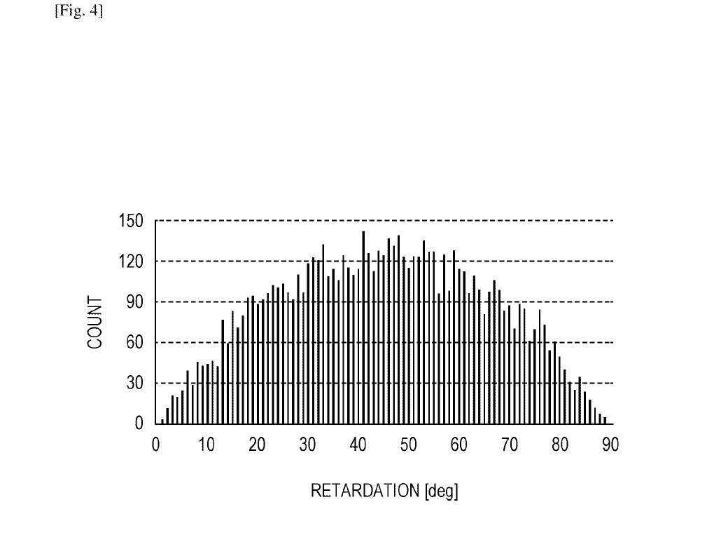

On the other hand, in a case where the resolution of the shooting apparatus is high in comparison with the reflection at the ultrastructures, the non-uniformly polarized light is observed in a separated manner. As a result, the intensity ratio (A_{V}/A_{H}) of the polarized light observed is a non-uniform value at each pixel, as illustrated in FIG. 4. Accordingly, the retardation calculated by Expression (2) also is non-uniform at each pixel. This is far from a correct representation of the state of the object, since non-uniform local states are being calculated. Note that even in cases of non-uniform retardation, spatially averaging retardation values approximates a constant value (δ=45°). A value obtained by spatially averaging retardation values (average value) is an example of a value exhibiting uniformity of retardation values.

Whether retardation is a constant value or non-uniform is the difference in relative resolution of the shooting apparatus, so the phenomenon itself is substantially the same. This is referred to as depolarization in the present embodiment, including non-uniform cases.

An example of a depolarizing region in a case where the object is an eye, is the RPE. The example in FIG. 3B is a region where the spatially averaged retardation of the region indicated by symbol A is 45°, and is a candidate region for depolarization. FIG. 3C illustrates an example of having extracted this candidate region for depolarization.

### Generating Retardation Map

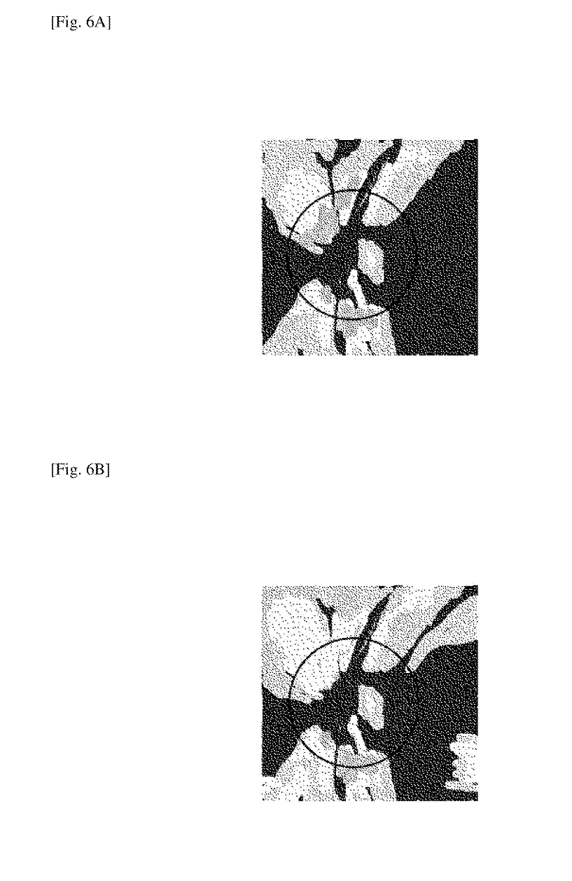

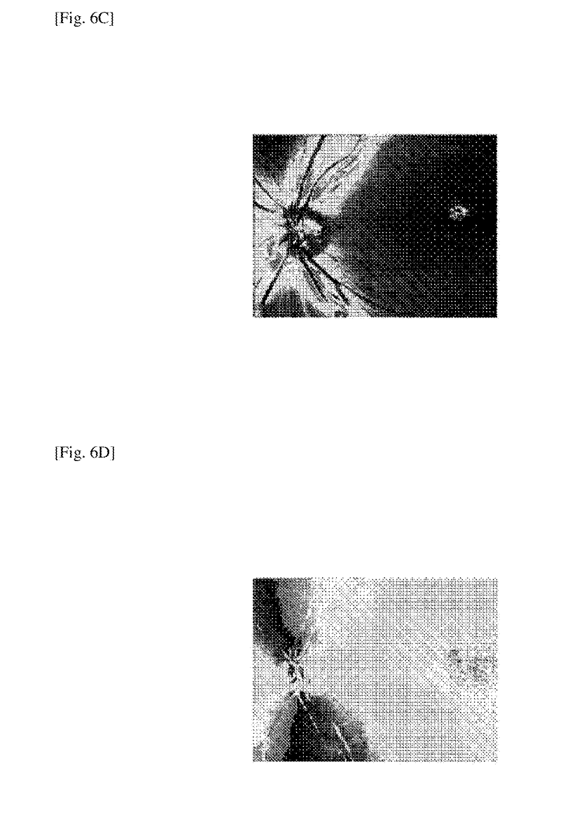

The signal processing unit 182, which is an example of an image generating unit that generates a retardation map in the planar direction of the retina, generates a retardation map from the retardation image obtained with regard to multiple B-scan images. The signal processing unit 182 detects the RPE in each B-scan image. The RPE has a nature of depolarization, so retardation distribution is inspected in each A-scan image in the depth direction, from the inner limiting membrane (ILM) over a range not including the RPE. The maximum value thereof is the representative value of retardation in the A-scan. The signal processing unit 182 performs the above processing on all retardation images, thereby generating a retardation map. FIG. 6A illustrates an example of a retardation map of the optic disc. FIG. 6C illustrates an example of a retardation map of the optic disc and macular area. Dark portions in intensity indicate a small value for the aforementioned ratio, and light portions in intensity indicate a great value for the aforementioned ratio. The retinal nerve fiber layer (RNFL) is a layer having birefringence at the optic disc. The retardation map is an image illustrating the difference in influence which the two polarized lights receive due to the birefringence of the RNFL and the thickness of the RNFL. Accordingly, the value indicating the aforementioned ratio is great when the RNFL is thick, and the value indicating the aforementioned ratio is small when the RNFL is thin. Thus, The thickness of the RNFL can be comprehended for the entire fundus from the retardation map, and can be used for diagnosis of glaucoma.

### Generating Birefringence Map

The signal processing unit 182 linearly approximates the value of retardation δ in the range of the ILM to the RNFL, in each A-scan image of the retardation images generated earlier, and determines the inclination thereof to be the birefringence at the position of the A-scan image on the retina. That is to say, the retardation is the product of distance and birefringence in the RNFL, so a linear relation is obtained by plotting the depth and retardation values in each A-scan image. Accordingly, this plot is subjected to linear approximation by the method of least squares, and the inclination is obtained, which is the value for birefringence of the RNFL in this A-scan image. This processing is performed on all retardation images that have been acquired, thereby generating a map representing birefringence, in the planar direction of the retina. FIG. 6B illustrates an example of a birefringence map of the optic disc. The birefringence map directly maps birefringence values, so even if the thickness of the RNFL does not change, change in the fiber structure thereof can be visualized as change in birefringence.

### Generating Orientation Image

Next, generating an orientation image, which is an example of an image indicating phase difference of polarized light, will be described. The signal processing unit 182 generates an orientation image from phases Φ_{H} and Φ_{V} of tomographic signals of mutually-orthogonal polarization components. A value θ in each pixel of the orientation image represents the direction of the optical axis as to measurement light, at the position of each pixel making up the tomographic image. This is calculated by Expression (4), from the phase difference ΔΦ(=Φ_{V}−Φ_{H}) of tomographic signals of mutually-orthogonal polarization components.

Θ=(π−ΔΦ)/2 Expression (4)

FIG. 6D illustrates an example of an orientation map of the optic disc and macular area. The orientation of the optical axis is due to anisotropy in the internal structure of the object. Anisotropy occurs along where nerve fibers run, for example. Accordingly, generating an orientation image enables the orientation of anisotropy of layers with birefringence to be comprehended. With regard to a case where interference light has been depolarized, the phases of the polarization components have no correlation (or are random), so the phase difference ΔΦ is a varied value. Orientation cannot be defined in regions with depolarization, so inaccurate values will be calculated if displayed as a tomographic image.

### Generating DOPU Image

Next, generating of a DOPU image will be described. The DOPU is a numerical value representing the uniformity of polarized light that is near 1 where polarization is maintained, but is smaller than 1 where depolarized. The signal processing unit 182, which is an example of a second computing unit, calculates a Stokes vector S for each pixel, from the obtained tomography signals A_{H} and A_{V}, and the difference ΔΦ of phase Φ_{V} and phase Φ_{H}(=Φ_{V}−Φ_{H}), by Expression (5).

$$\begin{matrix}[ Math . 2 ] & \\ S = ( \begin{matrix}I \\ Q \\ U \\ V\end{matrix} ) = ( \begin{matrix}{A}_{H}^{2} + {A}_{V}^{2} \\ {A}_{H}^{2} - {A}_{V}^{2} \\ 2 {A}_{H} {A}_{V} cos Δ φ \\ 2 {A}_{H} {A}_{V} sin Δ φ\end{matrix} ) & Expression ( 5 )\end{matrix}$$

The signal processing unit 182 sets a window for each B-scan image of a size around 70 μm in the main scanning of the measurement light and 18 μm in the depth direction. The signal processing unit 182 then averages each element of the Stokes vector (Stokes parameter) calculated for each pixel within each window in Expression (5), and calculates the DOPU in each window by Expression (6),

[Math.3]

DOPU=\sqrt{Qₘ²+Uₘ²+Vₘ²} Expression (6)

where Qₘ, Uₘ, and Vₘ are each values of the Stokes parameters Q, U, and V in each window averaged, and the intensity I normalized. Calculating uniformity within the window by DOPU enables stable evaluation of depolarization. Appropriately selecting the window size for DOPU allows evaluation for both cases where retardation is a constant value and a case where retardation is non-uniform, regarding depolarization. The region for averaging is determined by the window size, and can be decided taking into consideration the object and shooting apparatus resolution, pixel size, and so forth.

The signal processing unit 182 performs this processing on the depolarization candidate regions described below, thereby generating a DOPU image (also referred to as a tomographic image indicating the uniformity of polarized light) of the macular area, illustrated in FIG. 3D. The gradation bar to the right side in FIG. 3D indicates the value of DOPU in a range of 0 through 1. Portions that are light in intensity indicate that polarization is uniform, and portions that are dark in intensity indicate that polarization is non-uniform.

Next, description will be made regarding a method for extracting depolarization regions from DOPU values, performed by the signal processing unit 183 that is an example of a second extracting unit. The RPE has a nature of depolarizing in the structure in the retina, so portions in a DOPU that correspond to the RPE are smaller in value than other regions. Accordingly, depolarization regions can be extracted using a DOPU value as a threshold value. The threshold value changes depending on the pixel size of the measurement apparatus and the way in which the window is set, but can be decided by measuring the object beforehand. For example, a threshold of 0.75 may be set. Of the two-layer depolarization candidate region in FIG. 3D, the lower layer region that is darker in intensity (region B) corresponds to the RPE that is the depolarization region. The upper layer corresponds to the lower region of the ellipsoid zone (EZ) and is extracted as a depolarization candidate region from the retardation values, but can be determined to not be a depolarization layer by DOPU calculation, since the degree of depolarization is low. A DOPU image is a visualization of layers where depolarization occurs, such as at the RPE or the like, so even in cases where the RPE is deformed due to disease or the like, an image of the RPE can be formed in a sure manner by change in luminance. FIG. 3E illustrates an example of RPE extracted. The dark region (region B) in FIG. 3E corresponds to the RPE.

### Method for Extracting Candidate Region for Depolarization

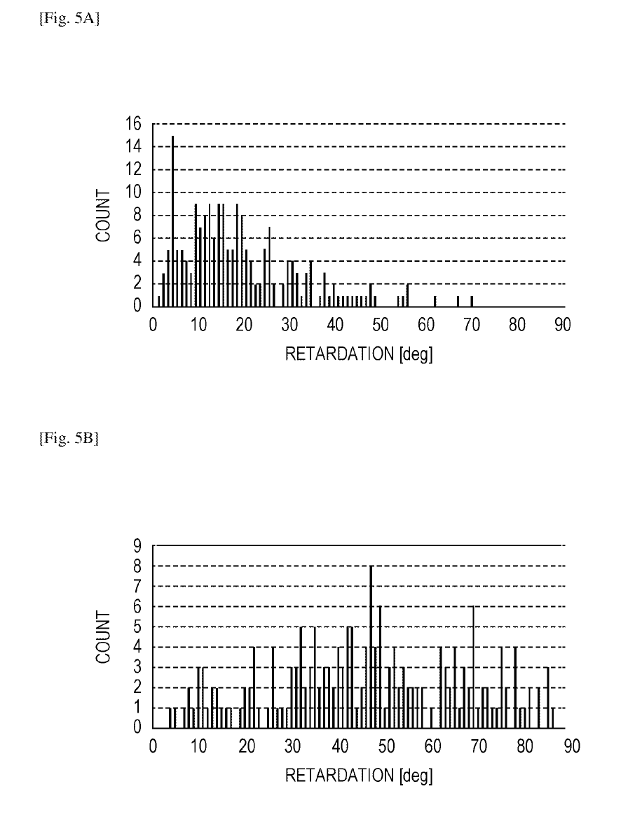

Description will be made regarding a method for using retardation values in the extracting of a candidate region for depolarization. Extracting of the candidate region for depolarization is performed by the signal processing unit 182, which is an example of a first extracting unit. Generally, in a case where the object is a human eye, retardation is smaller than 45°, so candidate regions for depolarization can be extracted using this characteristic. If there is no depolarization, i.e., in a case where retardation is maintained, a distribution (peak) reflecting the polarization properties of the tissue of the object is exhibited. On the other hand, in a case where there is depolarization, the value is constant (45°) or the values are varied from pixel to pixel, averaging out at approximately 45°. Whether the retardation is constant or non-uniform depends on difference in the relative resolution of the shooting apparatus. If the resolution of the shooting apparatus is lower in comparison with reflection at the ultrastructures, non-uniform polarization is averaged and observed, so bias in the polarized light is canceled out. With no bias in the polarized light, the intensity ratio of the polarization components is equal (A_{V}=A_{H}). Accordingly, retardation δ is a constant value. On the other hand, in a case where the resolution of the shooting apparatus is higher in comparison with reflection at the ultrastructures, non-uniform (random) polarized light is observed in a separated manner, so the intensity ratio (A_{V}/A_{H}) of the polarized light components is a non-uniform value. In this case, the retardation at each pixel also is non-uniform. Non-uniformity of retardation can be evaluated by setting a predetermined window and making evaluation based on average value and variance, in the same way as in DOPU calculation. FIG. 5A is a distribution example where a window is set in the RNFL, and FIG. 5B in the RPE layer. It can be seen in FIG. 5A that retardation is distributed in a biased manner at or below 45°. On the other hand, the variance in the window is great in FIG. 5B, from 0° to 89°, so it can be judged that polarization has been canceled.

From the above, in can be seen that average values of retardation, for example can be used as an index to determined depolarization. In a case where retardation is ideally randomly distributed, the average value of retardation will be 45°, but average is calculated within a set window, and accordingly contains error.

Accordingly, a value Rth of retardation to be used as determination index is set, based on the concept of confidence interval. Rth is found from Expression (7).

[Math.4]

Rₜₕ=μ±T×σ/\sqrt{N} Expression (7)

where μ represents the average value of retardation within a window, σ represents standard deviation, N represents degree of freedom, and T represents a t value.

Table 1 illustrates the average values of retardation, and standard deviation, for a window set for the four layers of the NFL, the inner plexiform layer (IPL), the RPE, and the choroid. AS a result of diligent study by the present inventors, it was found that the average value μ of retardation within a window set to the RPE layer was 44.9°, and the standard deviation σ was 23°.

TABLE 1

| | Average value | Standard deviation |
|---|---|---|
| Retinal layer | (deg) | (deg) |
| Retinal nerve fiber layer (RNFL) | 18.9 | 13.9 |
| Inner plexiform layer (IPL) | 16.8 | 10.5 |
| Retinal pigment epithelium (RPE) | 44.9 | 24.2 |
| Choroid | 33.4 | 18 |

A numeral 89, obtained by subtracting 1 from the number of the pixels in the window is set as the degree of freedom N, and 3.29 is set as the t value guaranteeing the reliability standard of 99.9%, and substituted into Expression (7), yielding 44.9±8.0° as Rth. Note however, in a case of setting the same window to the RPE layer, the average value of retardation will fall within a range of values from 36.9° to 52.9° with a 99.9% probability.

In light of the fact that the retardation of the object normally is smaller than 45°, a depolarization candidate may be extracted in a case where the average value of retardation within the window is higher than 36.9°, for example. When shooting in actual practice, a candidate region for depolarization is extracted in a case where the average value is 35° or above in the present embodiment, giving extra consideration to differences among objects and error due to the shooting environment.

The confidence interval is dependent on the apparatus environment and window size, so the value of Rth is an item to be set in design. Accordingly, Rth may be set as appropriate in accordance with the apparatus environment and size of the window being set.

Also, in light of the fact that the retardation normally is smaller than 45°, the number of retardations exceeding 45° in the window may be used as depolarization candidates. In a case where depolarization is occurring, ideally, half of the number of pixels included in the window will exhibit retardation of 45° or higher. For example, in a case of setting a window of 90 pixels, the number of pixels exhibiting retardation of 45° or higher will ideally be 45. Accordingly, a candidate for depolarization may be set in a case where the number of retardations 45° or higher is 40 pixels or more, for example.

### Segmentation

The signal processing unit 182 performs segmentation of the tomographic image using the above-described luminance image. The signal processing unit 182 applies a median filter and a Sobel filter to the tomography image to be processed, and creates images by each (hereafter also referred to as “median image” and “Sobel image”). Next, a profile is created for each A scan, from the created median image and Sobel image. A luminance value profile is created from the median image, and a gradient profile is created from the Sobel image. The signal processing unit 182 detects peaks in the profile created from the Sobel image. The signal processing unit 182 references the profile of the median image corresponding to nearby the detected peaks or between the peaks, thereby extracting the boundaries of the regions of the retina layer. Further, the signal processing unit 182 can measure the layer thicknesses in the A scan line direction, and create a thickness map of the layers, which are in the plane direction of the retina. Further, birefringence can be obtained from retardation, using the results of segmentation. The rate of change of retardation in the depth direction (i.e., inclination) corresponds to birefringence.

### Processing of Extracting Depolarization Region

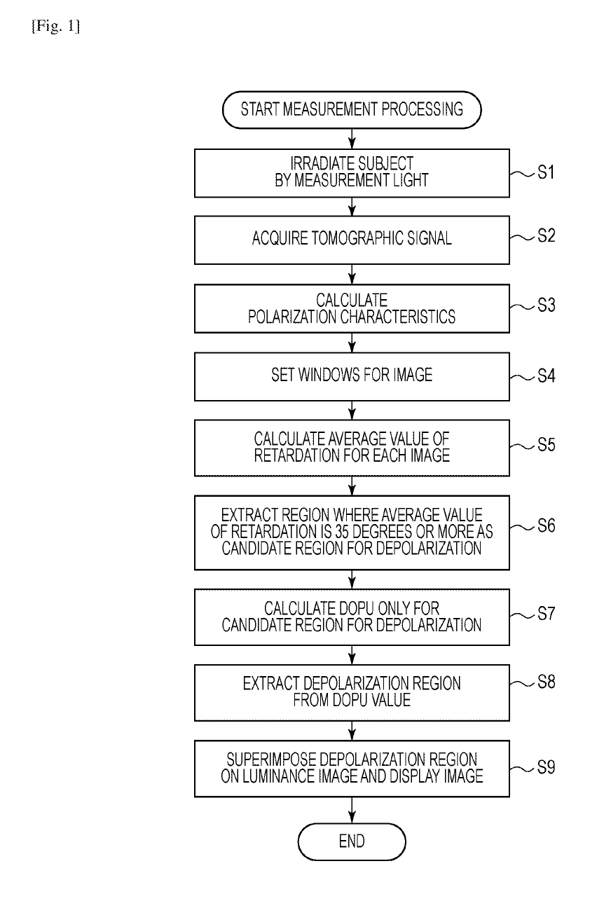

Next, the flow of shooting according to the present embodiment will be described with reference to FIG. 1. The flowchart in FIG. 1 is a flowchart illustrating measurement processing by the shooting apparatus. When the user selects the measurement mode, by operating a measurement start button (omitted from illustration) displayed on the display unit 184 or a measurement start button physically provided to the main unit, for example, the control unit 180 accepts a measurement start instruction, sets the operation mode to the measurement mode, and starts measurement.

In S1, the driving control unit 181 irradiates the object by measurement light.

Next, in S2, the control unit 180 acquires interference signals from the line cameras 129 and 133, and by performing signal processing obtains tomographic signals A_{H} and A_{V} corresponding to the object. The tomographic signals A_{H} and A_{V} contain information of the polarization characteristics of the object.

In S3, the polarization characteristics of the object are calculated. The polarization characteristics of the object to be calculated include at least retardation. FIG. 3B illustrates an example calculating retardation as a polarization characteristic.

In S4, the signal processing unit 182 sets windows for the entire region of the retardation tomographic image. The size of the windows being set may be decided taking into consideration the object and shooting apparatus resolution, pixel size, and so forth. For example, the size may be around 70 μm in the main scanning of the measurement light and 18 μm in the depth direction, for example, in the same way as with DOPU.

The average value of retardation is then calculated by the signal processing unit 182 in S5, for each window set in S4.

Next, in S6 the signal processing unit 182 extracts windows where the average value of retardation calculated in S5 is a threshold value or above, e.g., 35° or above as candidate regions for depolarization. FIG. 3C illustrates an example of extracted candidate regions for depolarization.

Then in step S7, the signal processing unit 182 calculates the DOPU for the windows extracted in S6 as candidate regions for depolarization.

In S8, the signal processing unit 182 extracts depolarization regions. Extracting of depolarization regions can be performed using DOPU. Regions where the value of DOPU is equal to or smaller than a threshold (e.g., regions where the DOPU is 0.75 or lower) may be taken as depolarization regions. FIG. 3D shows an example of DOPU acquisition.

Finally, in S9, the control unit 183, which is an example of a display control unit, superimposes the depolarization regions on the luminance image. The superimposed image is displayed on the display unit 184, and the measurement processing ends. An example of superimposing extracted depolarization regions on a luminance image is shown in FIG. 3E.

### Comparative Example

International Publication No. 2012/0265059 is known literature that describes detecting depolarization without using Stokes vector calculation. This literature discloses a system that acquires signals while changing polarization of measurement light, and identifies regions where intensity information of detected light does not change, thereby detecting depolarization. This system performs signal acquisition while changing the polarization state of the measurement light, and accordingly needs a control system to acquire signals while changing polarization. This system also other problems due to multiple sets of signal data with changed polarization state of measurement light being necessary for each shooting position, resulting in an increased amount of calculation and data in signal processing, taking long periods of time for analysis, and so forth.

### Other Embodiments

Although description has been made above that emitted light that has been emitted from the light source 101 is adjusted into perpendicularly polarized light at the polarization controller 103, the emitted light may be adjusted into linearly polarized light of another orientation, such as horizontally polarized light or the like. In the case of using another orientation, the angle of the wave plate and the calculation expression may be changed correspondingly.

Also, although the shooting apparatus has been described in the above embodiment as being a spectral-domain polarization-sensitive OCT (SD-PS-OCT) apparatus, the shooting apparatus may be applied to a swept source PS-OCT apparatus or time-domain OCT apparatus. Further, the shooting apparatus may be applied to other PS-OCT apparatuses, such as a PS-OCT apparatus using a system where polarization of measurement light is modulated using an electro-optic modulator (EOM), or the like.

The object of the shooting apparatus is not restricted to that described in the embodiment above. It is sufficient for the shooting apparatus to be an OCT apparatus that measures polarization characteristics of an object, and may be an OCT apparatus that measures biological objects other than eyes, such as skin, internal organs, blood vessels, teeth, and so forth, or an OCT that measures polarization characteristics of specimens other than biological objects. The shooting apparatus may be an endoscope.

The present invention may also be realized by executing the following processing. That is to say, software (program) realizing the functions of the above-described embodiment is supplied to a system or an apparatus via a network or any of various types of storage mediums. A computer (or control processing unit (CPU) or microprocessor unit (MPU) or the like) of the system or apparatus then reads out and executes the program. For example, acquisition of tomographic signals (S1 and S2 in FIG. 1) and post-processing (S3 through S9 in FIG. 1) may be performed separately.

According to the above embodiments, polarization OCT images can be clearly displayed even if there are depolarization regions in the object.

Although the present invention has been described regarding preferred embodiments, the present invention is not limited to any particular embodiment. Various types of modifications and alterations may be made without departing from the scope of the preset invention set forth in the Claims.

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-215220, filed Oct. 30, 2015, which is hereby incorporated by reference herein in its entirety.

1. An image processing apparatus that processes a plurality of tomographic signals corresponding to light of mutually different polarization, acquired from an object using light interference, the image processing apparatus comprising:
a first computing unit configured to compute distribution of retardation values of the object, based on the plurality of tomographic signals;
a first extracting unit configured to extract candidate regions for depolarization regions of the object in a retardation image of the object, based on the calculated distribution of retardation values;
a second computing unit configured to compute distribution of a value indicating uniformity of polarized light in the extracted candidate regions, based on the plurality of tomographic signals in the extracted candidate regions; and
a second extracting unit configured to extract depolarization regions of the object in an image indicating uniformity of polarized light in the extracted candidate regions, based on distribution of the calculated value indicating uniformity of polarized light.

2. The image processing apparatus according to claim 1, wherein the first extracting unit extracts a region, where distribution of the calculated retardation values in the retardation image is 35° or greater, as the candidate region.

3. The image processing apparatus according to claim 1, wherein the first extracting unit extracts a region, where an average value of distribution of the calculated retardation values in windows set in the retardation image is a threshold value or greater, as the candidate region.

4. The image processing apparatus according to claim 1, further comprising:
an image generating unit configured to generate a map of the object in a planar direction, based on the extracted depolarization regions.

5. The image processing apparatus according to claim 1, further comprising:
an image generating unit configured to identify a representative value of retardation in the depth direction at a plurality of positions in the planar direction of the object, based on the plurality of tomographic signals, and generate a retardation map using the identified representative values.

6. An image processing apparatus that processes a plurality of tomographic signals corresponding to light of mutually different polarization, acquired from an object using light interference, the image processing apparatus comprising:
a first extracting unit configured to extract candidate regions for depolarization regions of the object in an image indicating phase difference of polarized light of the object, based on a value indicating phase difference of polarized light of the object obtained using the plurality of tomographic signals; and
a second extracting unit configured to extract depolarization regions of the object in an image indicating uniformity of polarized light in the extracted candidate region, based on a value indicating uniformity of polarized light in the extracted candidate region, obtained using the plurality of tomographic signals of the extracted candidate regions.

7. The image processing apparatus according to claim 6, wherein the first extracting unit extracts a region, where the computed value indicating the phase difference of polarized light in the image indicating phase difference of polarized light is 35° or greater, as the candidate region.

8. The image processing apparatus according to claim 1, wherein the second extracting unit extracts a region where a value indicating uniformity of polarized light that has been calculated is equal to or below a threshold value, as the depolarization region.

9. The image processing apparatus according to claim 1, further comprising:
a display control unit configured to display, on a display unit, the extracted depolarization regions superimposed on a tomographic luminance image of the object generated based on the plurality of tomographic signals.

10. The image processing apparatus according to claim 6, wherein the object is an eye to be examined.

11. The image processing apparatus according to claim 1, communicably connected to an optical interference tomographic apparatus having a detecting unit configured to detect light of mutually different polarization, obtained by dividing interference light obtained by interference between return light from the object irradiated by measurement light an reference light corresponding to the measurement light,
wherein the plurality of tomographic signals are acquired based on the detected light of mutually different polarization.

12. The image processing apparatus according to claim 1, wherein the object is an eye to be examined.

13. (canceled)

14. (canceled)

15. (canceled)

16. An image processing method of processing a plurality of tomographic signals corresponding to light of mutually different polarization, acquired from an object using light interference, the method comprising:
computing retardation values of the object, based on the plurality of tomographic signals;
extracting candidate regions for depolarization regions of the object in a retardation image of the object, based on the calculated retardation values;
computing distribution of a value indicating uniformity of polarized light in the extracted candidate regions, based on the plurality of tomographic signals in the extracted candidate regions; and
extracting depolarization regions of the object in an image indicating uniformity of polarized light in the extracted candidate regions, based on distribution of the calculated value indicating uniformity of polarized light.

17. (canceled)

18. An image processing method of processing a plurality of tomographic signals corresponding to light of mutually different polarization, acquired from an object using light interference, the method comprising:
extracting candidate regions for depolarization regions of the object in an image indicating phase difference of polarized light of the object, based on a value indicating phase difference of polarized light of the object obtained using the plurality of tomographic signals; and
extracting depolarization regions of the object in an image indicating uniformity of polarized light in the extracted candidate region, based on a value indicating uniformity of polarized light in the extracted candidate region, obtained using the plurality of tomographic signals of the extracted candidate regions.

19. A program to cause a computer to execute the image processing method according to claim 16.

20. A program to cause a computer to execute the image processing method according to claim 19.
